SEQUENCE LISTING (49 sequences)

SEQ ID NO: 1 - protein, 13 aa, Homo sapiens
AQQKSLLQQLLTE

SEQ ID NO: 2 - protein, 25 aa, Homo sapiens
QEPVSPKKKENALLRYLLDKDDTKD

SEQ ID NO: 3 - protein, 24 aa, Homo sapiens
SPSSHSSLTERHKILHRLLQEGSP

SEQ ID NO: 4 - protein, 24 aa, Homo sapiens
QKPTSGPQTPQAQQKSLLQQLLTE

SEQ ID NO: 5 - protein, 15 aa, Homo sapiens
AEEPSLLKKLLLAPA

SEQ ID NO: 6 - protein, 24 aa, Homo sapiens
SGNLVPDAASKHKQLSELLRGGSG

SEQ ID NO: 7 - protein, 24 aa, Homo sapiens
GHGEDFSKVSQNPILTSLLQITGN

SEQ ID NO: 8 - protein, 20 aa, Rattus rattus
PCQGSASHPTILYTLLSPGP

SEQ ID NO: 9 - protein, 20 aa, Rattus rattus
VAEAPVPSILKKILLEEPNS

SEQ ID NO: 10 - protein, 25 aa, Homo sapiens
GHSFADPASNLGLEDIIRKALMGSF

SEQ ID NO: 11 - protein, 27 aa, Homo sapiens
QAVQEHASASTNMGLEAIIRKALMGKY

SEQ ID NO: 12 - protein, 21 aa, Homo sapiens
ASTNMGLEAIIRKALMGKYDQ

SEQ ID NO: 13 - protein, 15 aa, Rattus rattus
ASHPTILYTLLSPGP

SEQ ID NO: 14 - protein, 17 aa, Rattus rattus
APVPSILKKILLEEPNS

SEQ ID NO: 15 - protein, 15 aa, Rattus rattus
ASQGRLARILLMAST

SEQ ID NO: 16 - protein, 15 aa, Homo sapiens
QWQGSILYNMLMSAK

SEQ ID NO: 17 - protein, 15 aa, Homo sapiens
PRQGSILYSMLTSAK

SEQ ID NO: 18 - protein, 15 aa, Homo sapiens
PRQGSILYSLLTSSK

SEQ ID NO: 19 - protein, 15 aa, Homo sapiens
SQTSHKLVQLLTTTA

SEQ ID NO: 20 - protein, 15 aa, Homo sapiens
TERHKILHRLLQESS

SEQ ID NO: 21 - protein, 15 aa, Homo sapiens
SKDHQLLRYLLDKDE

SEQ ID NO: 22 - protein, 13 aa, Homo sapiens
AQQKSLLQQLLTE

SEQ ID NO: 23 - protein, 15 aa, Homo sapiens
SKGQTKLLQLLTCSS

SEQ ID NO: 24 - protein, 15 aa, Homo sapiens
KEKHKILHRLLQDSS

SEQ ID NO: 25 - protein, 14 aa, Homo sapiens
KENALLRYLLDKDD

SEQ ID NO: 26 - protein, 15 aa, Homo sapiens
SKGHKKLLQLLTCSS

SEQ ID NO: 27 - protein, 15 aa, Homo sapiens
QEKHRILHKLLQNGN

SEQ ID NO: 28 - protein, 15 aa, Homo sapiens
KENNALLRYLLDRDD

SEQ ID NO: 29 - protein, 15 aa, Homo sapiens
VSQNPILTSLLQITG

SEQ ID NO: 30 - protein, 15 aa, Homo sapiens
KNIHPMLMNLLKDNP

SEQ ID NO: 31 - protein, 15 aa, Homo sapiens
ASKHKQLSELLRGGS

SEQ ID NO: 32 - protein, 15 aa, Homo sapiens
AEEPSLLKKLLLAPA

SEQ ID NO: 33 - protein, 15 aa, Homo sapiens
RRPCSELLKYLTTND

SEQ ID NO: 34 - protein, 15 aa, Homo sapiens
VDELSLLQKLLLATS

SEQ ID NO: 35 - protein, 15 aa, Homo sapiens
WAEFSILRELLAQDV

SEQ ID NO: 36 - protein, 15 aa, Homo sapiens
PREGSSLHKLLTLSR

SEQ ID NO: 37 - protein, 15 aa, Homo sapiens
QQQAQQLYSLLGQFN

SEQ ID NO: 38 - protein, 15 aa, Homo sapiens
RETSEKFKLLFQSYN

SEQ ID NO: 39 - protein, 15 aa, Homo sapiens
TLTSPLLVNLLQSDI

SEQ ID NO: 40 - protein, 15 aa, Homo sapiens
REAPTSLSQLLDNSG

SEQ ID NO: 41 - protein, 15 aa, Homo sapiens
KQDSTLLASLLQSFS

SEQ ID NO: 42 - protein, 15 aa, Homo sapiens
SKSFNVLKQLLLSEN

SEQ ID NO: 43 - protein, 15 aa, Homo sapiens
NADDAILRELLDESQ

SEQ ID NO: 44 - protein, 15 aa, Homo sapiens
NLPPAALRKLLRAEP

SEQ ID NO: 45 - protein, 15 aa, Homo sapiens
FAGDEVLVQLLSGDK

SEQ ID NO: 46 - protein, 13 aa, Homo sapiens
HSSRLWELLMEAT

SEQ ID NO: 47 - protein, 13 aa, Homo sapiens
YRGAFQNLFQSVR

SEQ ID NO: 48 - protein, 13 aa, Homo sapiens
ASSSWHTLFTAEE

SEQ ID NO: 49 - protein, 12 aa, Homo sapiens
QPKHFTELYFKS

(19) United States
(12) Patent Application Publication
(10) Pub. No.: US 2007/0111265 A1
(43) Pub. Date: May 17, 2007

(54) Ligands for mineralocorticoid receptor (MR) and methods for screening for or designing MR ligands

(75) Inventors: Huaqiang Eric Xu, Grand Rapids, MI (US); Yong Li, Grand Rapids, MI (US)

Correspondence Address: PRICE HENEVELD COOPER DEWITT & LITTON, LLP, 695 KENMOOR, S.E., P O BOX 2567, GRAND RAPIDS, MI, 49501, US

(73) Assignee: Van Andel Research Institute

(21) Appl. No.: 11/598,933

(22) Filed: Nov. 14, 2006

Related U.S. Application Data
(60) Provisional application No. 60/737,054, filed on Nov. 15, 2005.

(51) Int. Cl. G01N 33/567; G06F 19/00; A61K 31/573

(52) U.S. Cl. 435/7.2; 514/179; 702/19

(57) ABSTRACT

The inventors disclose a 1.95 Å crystal structure of the MR ligand binding domain containing a single C808S mutation bound to a corticosterone and the fourth LXXLL motif of steroid receptor coactivator-1 (SRC1-4). The inventors demonstrate that SRC1-4 is the most potent MR-binding motif and mutations that disrupt the MR/SRC1-4 interactions abolish the ability of the full-length SRC1 to coactivate MR. The structure also reveals a compact steroid binding pocket with a unique topology that is primarily defined by key residues of helices 6 and 7. Also described are novel ligands for MR, methods for screening for and designing novel MR ligands, and methods for treating MR-related diseases.

### CROSS-REFERENCE TO RELATED APPLICATION

This application claims the benefit under 35 U.S.C. § 119(e) of U.S. Provisional Application No. 60/737,054, entitled Ligands for Mineralocorticoid Receptor (MR) and Methods for Screening for or Designing MR Ligands, filed on Nov. 15, 2005, the entire disclosure of which is hereby incorporated herein by reference.

### FIELD OF THE INVENTION

This invention relates to the discovery of ligands for the classic steroid hormone receptor named mineralocorticoid receptor (MR).

### BACKGROUND OF THE INVENTION

Mineralocorticoid receptor (MR) is a member of the classic steroid hormone receptors that include glucocorticoid receptor (GR), androgen receptor (AR), progesterone receptor (PR), and estrogen receptor (ER) (Funder, 1997). These receptors are hormone-activated transcriptional factors that regulate a wide variety of physiological processes ranging from organ development and differentiation to mood control and stress response (Beato et al., 1995). MR, in particular, is required for the maintenance of electrolyte homeostasis and blood pressure (Funder, 1997). Mutations in MR have been associated with early onset of severe hypertension and pregnancy induced hypertension (Geller et al., 2000). As such, MR is an important drug target, which is underscored by the clinical use of two MR antagonists, spironolactone and eplernone, in the treatment of hypertension and heart failure (Funder, 2003). However, the application of these MR antagonists is limited by potential side effects associated with the cross reactivity with other steroid receptors or by the low binding affinity to MR (Baxter et al., 2004). Thus, discovery of a highly potent and selective MR antagonist remains a major interest of pharmaceutical research.

The human MR contains 984 amino acids that are organized into three functional domains: an N-terminal activation function-1 domain (AF-1), a middle DNA binding domain (DBD) and a C-terminal ligand binding domain (LBD) (Arriza et al., 1987). The functional activity of both the AF-1 domain and the DBD are controlled by hormone binding to the LBD (Rogerson and Fuller, 2003). In addition to ligand binding, the MR LBD contains an activation function-2 domain (AF-2) that is regulated by hormone binding, as well as sequence motifs that mediate the functions of heat-shock proteins (HSPs), nuclear translocation, and recruitment of transcriptional co-factors [reviewed in (Galigniana et al., 2004)]. Thus, the MR LBD is the key regulatory domain of the receptor, whose functions require the structural integrity of the whole LBD.

The physiological hormone for MR is aldosterone in humans and corticosterone in rodents (Funder et al., 1988). Both steroids bind to human MR with high affinity. In the absence of the hormone, MR exists predominantly in the cytoplasm in a complex with heat shock chaperones (Bruner et al., 1997). As it is the case for GR, the association of steroid receptors with HSPs not only keeps the receptor inactive in the absence of hormone but also maintains the receptor structure in a conformation that permits high affinity ligand binding (Picard et al., 1990). Hormone binding induces conformational changes in the MR LBD that initiate a cascade of events, including the release of chaperone proteins, nuclear localization and DNA binding (Galigniana et al., 2004). As such, hormone binding to the MR LBD is the critical step that activates the receptor.

Following the hormone binding, the transcriptional function of MR is mediated through the recruitment of specific coactivators to the MR-regulated genes. Coactivators such as steroid receptor coactivator-1 (SRC1, (Onate et al., 1995)) and transcriptional intermediary factor 2 (TIF2, also known as GRIP1/SRC2, (Hong et al., 1997; Voegel et al., 1998)) contain multiple LXXLL motifs to interact with nuclear receptors. Crystal structures of various LBD/LXXLL motif complexes reveal a common charge clamp mechanism, in which a glutamate residue from the AF-2 helix and a lysine residue from helix 3 mediate capping interactions with both ends of the two turn α-helix formed by the LXXLL motifs. MR LBD also contains the conserved charge clamp residues and presumably recruits coactivators through its interactions with LXXLL motifs (Hong et al., 1997; Hultman et al., 2005). However, there are many coactivators and each contains multiple LXXLL motifs. The precise repertoire of coactivators and the mode of their assembly with MR remain unexplored.

Endogenous steroid hormones such as corticosterone and progesterone share closely related chemical structures yet mediate dramatically different physiology through the binding to their cognate receptors. Our understanding at the molecular level of how steroid receptors achieve their hormone specificity has been enhanced by the previous structures of hormone complexes of GR, AR, PR and ER (Bledsoe et al., 2002; Matias et al., 2000; Shiau et al., 1998; Williams and Sigler, 1998). These structures reveal a general binding mode of steroid hormones within the pocket of the LBD and identify key residues that interact with specific steroid functional groups. Based on these structural observations, it has been proposed that steroid selectivity is achieved by matching the shape and hydrogen bonds between ligands and the ligand binding pocket of the receptors (Bledsoe et al., 2002). However, the molecular basis that determines the MR hormone selectivity remains uncertain in the absence of a MR structure.

### SUMMARY OF THE INVENTION

The inventors report herein a 1.95 Å crystal structure of the MR ligand binding domain containing a single C808S mutation, bound to corticosterone and the fourth LXXLL motif of steroid receptor coactivator-1 (SRC1-4). Through a combination of biochemical and structural analyses, the inventors demonstrate that SRC1-4 is the most potent MR-binding motif and mutations that disrupt the MR/SRC1-4 interactions abolish the ability of the full-length SRC1 to coactivate MR. The structure also reveals a compact steroid binding pocket with a unique topology that is primarily defined by key residues of helices 6 and 7. Mutations swapping a single residue at position 848 from helix H7 between MR and glucocorticoid receptor switch their hormone specificity. The invention provides critical insights into the molecular basis of hormone binding and coactivator recognition by MR and related steroid receptors.

The present invention provides a method for designing novel ligands for mineralocorticoid receptor (MR). In a preferred embodiment, the present invention provides a method for designing novel ligands that form direct hydrogen bonds with MR residue S810. The present invention also comprises a method for screening for MR ligands and/or coactivators.

The inventors disclose the crystal structure of the MR ligand binding domain with key structural features that define specific recognition of hormones and co-activators by MR, and provide a rational template for designing selective and potent ligands of MR for the treatment of various diseases including hypertension and heart failure.

The identification of agonistic or antagonistic MR ligands also will provide a chemical tool to probe biology and physiology of this receptor using various known methods.

### BRIEF DESCRIPTION OF THE DRAWINGS

FIG. 1. Purification, Characterization and Crystallization of the MR LBD

(A) Purification of the MR LBD bound to corticosterone. The proteins shown are crude extract (lane 1), the GST column flow through (lane 2), the GST column elute (lane 3), the sample after thrombin digestion (lane 4) and final purified protein (lane 5). The molecular weight markers are shown in lane M.

(B) Binding of various peptides containing coactivator or corepressor motifs to the purified MR LBD/corticosterone complex as measured by AlphaScreen assays. The background reading with the MR LBD alone is less than 200.

(C) Relative binding affinity of various peptide motifs to the MR LBD in the presence of 20 nM of corticosterone or aldosterone as determined by peptide competitions which various unlabeled peptides (500 nM) are used to compete off the binding of the SRC2-3 LXXLL motif to MR. The cofactors that contain a pair of LXXLL motifs with strong binding affinity to MR are boxed. All peptides have identical length of 15 residues except for SRC1-4 motif, which terminates at position +7 relative to the first leucines (L+1) in the LXXLL motif, and for the AR peptides and the corepressor motifs, which are longer than the coactivator motifs. Sequences of peptides are listed in experimental procedures.

(D) Crystals of the MR/corticosterone/SRC1-4 complex.

The results in panels C-D are the average of experiment performed in triplicate, with error bars showing SDs.

FIG. 2. Overall Structure of the MR/corticosterone/SRC1-4 complex

(A-B) Two 900 views of the MR/corticosterone/SRC1-4 complex in ribbon representation. MR is colored in gold with its charge clamp residues colored in red (AF-2) and blue (end of H3). The SRC1-4 peptide is in yellow and the bound corticosterone is shown in ball & stick representation with carbon and oxygen atoms depicted in green and red, respectively. Key structural elements are noted including β-6 following with the LYFH motif near the C-terminal end.

(C) Sequence alignment of the human MR LBD with other steroid hormone receptors (GR, AR, PR and ER). The secondary structural elements are boxed and annotated below the sequences, and the residues that form the steroid binding pockets are shaded in gray. The second charge clamp residues and K782, which comprise the two key structural features for the binding of SRC LXXLL motifs, are noted with stars, and the residues that determine MR/GR hormone specificity are labeled by arrows. The LYFH motifs near the C-terminal ends of oxosteroid receptors are underlined.

FIG. 3. Recognition of the SRC1-4 LXXLL Motif and Coactivator Assembly by MR

(A) Structure of the SRC1-4 LXXLL motif (green) is shown on the surface of the MR coactivator binding site.

(B) The binding mode of SRC1-4 to the MR LBD. MR is in light green and SRC1-4 is in yellow. The hydrogen bonds formed between MR and SRC1-4 are shown in arrows from hydrogen bond donors to acceptors. For residues Q-4 and Q-5, only Cα atoms are shown for clarity.

(C) A 2Fₒ-F_{c} electron density map (1.0σ) showing the structural stability of the SRC1-4 LLQQLL motif.

(D) Binding affinity of various coactivator LXXLL motifs to the purified MR/corticosterone complex as determined by IC50 values from peptide competition experiments using AlphaScreen assays. The numbering scheme of the LXXLL motifs is shown on the top of the sequences.

(E) Purification of PGC1α-(1+2) and SRC2-(2+3). The proteins shown are PGC1α-(1+2) (lane 1) and SRC2-(2+3) (lane 2).

(F) Binding affinity of SRC2-(2+3), SRC2-2, SRC2-3 and SRC2-(M2+3) to the purified MR/corticosterone complex as determined by IC50 values from peptide competition experiments using AlphaScreen assays. SRC2-2 and SRC2-3 are peptides shown in FIG. 3D. SRC2-(2+3) and SRC2-(2+3) are SRC2 protein fragments containing 2nd and 3rd LXXLL motifs. The 2nd LXXLL motif of SRC2 was mutated to LXXAA in SRC2-(M2+3).

(G) Binding affinity of PGC1α-(1+2), PGC1α-1 and PGC1α-2 to the purified MR/corticosterone complex as determined by IC50 values from peptide competition experiments.

FIG. 4. SRC1 Potentiates Transcription by MR through the SRC1-4 Motif

(A) A schematic representation of wild type (WT) and mutated SRC1 coactivator showing the locations of the four LXXLL motifs.

(B) The SRC1-4 motif is required to potentiate MR-mediated transcription. 50 ng Gal4-MR LBD was cotransfected with pG5Luc and increasing amount (ng) of SRC1 wild-type and 3 LXXAA mutant forms for LXXLL motifs. The cells were treated with and without 10 nM corticosterone. The dashed line indicates the basal level of activation without exogenous SRC1.

(C) Mammalian two-hybrid interaction of SRC1 with MR. GAL4-DBD were fused with the SRC1-4 motif (SRC1-4, residues 1240-1441) and two mutated forms of SRC1-4 [SRC1-4(E1441K), corresponding to E+7K mutation of the SRC1-4 motif, and SRC1-M4 (L1438A/L1439A), corresponding to the LXXAA mutation of the SRC1-4 motif], respectively. VP16 were fused with MR LBD and MR LBD (K782E). The cells were cotransfected with GAL4 and VP16 fusion constructs and pG5Luc reporter. The cells were treated with 10 nM corticosterone.

(D) Binding of various peptides to the purified MR LBD (C808S) with wild type (WT) charge clamps or mutated charge clamps in the presence of corticosterone (100 nM) as measured by AlphaScreen assays. K785E and E796R: 1st charge clamp mutations; K791E and E796R: 2nd charge clamp mutations.

The results in panels B-D are the average of three experiments with error bars showing SDs.

FIG. 5. Recognition of Corticosterone by MR and ligand binding specificity of GR and MR.

(A) A 2Fo-Fc electron density map (2.2σ) showing the bound corticosterone and the surrounding MR residues.

(B) Schematic representation of MR/corticosterone interactions. Hydrophobic interactions are indicated by dashed lines and hydrogen bonds are indicated by arrows from proton donors to acceptors. Residues that make polar and non-polar interactions with ligand are colored in blue and white, respectively.

(C & D) Overlays of the MR/corticosterone structure with the GR/dexmethasone structure, where MR is in light green and GR is in dark green. The key residues that determine MR and GR selectivity are noted with MR ligand binding pocket shown in red surface while GR ligand binding pocket shown in blue surface. MR residues are labeled in red and GR in blue. The arrows indicate the relative shift of the MR residues S843 and L848 with the corresponding GR residues P637 and Q642.

(E-H) Effects of mutations of key residues on hormone specificity between MR and GR. Dose-response curves for induction of luciferase activity by MR, MRL848Q and MRL848Q/S843P (E & F), GR, GRQ642L and GRQ642L/P637S (G & H) in response to cortisol and corticosterone respectively. The estimated EC50 values are shown with dotted lines. The results are the average of three experiments with error bars showing SDs.

FIG. 6. Molecular Basis for the Specificity of Steroid Hormones

(A) Chemical structures of the steroid hormones. The numbering of the rings and key atoms are noted.

(B) Summary of structural comparison steroid hormone receptors, including the pocket sizes, sequence homology (% of identity in the LBDs), and the RMSD values of the Cα atoms of the core LBD when MR was super-positioned with GR, PR, AR, and ER, respectively.

(C and D) An overlapping comparison of the MR structure (light green) with the structure of AR (panel C) and PR (panel D), where the hormones are shown in stick & ball and AR/PR are shown in dark green. The key residues that determine hormone selectivity are noted with MR ligand binding pocket shown in red surface while AR and PR ligand binding pocket shown in blue surface. MR residues are labeled in red, and AR and PR in blue.

### DETAILED DESCRIPTION OF THE PREFERRED EMBODIMENTS

The preferred embodiments of the present invention may be understood more readily by reference to the following detailed description of the specific embodiments and the Examples and Sequence Listing included hereafter.

The Sequence Listing filed with this application is contained on the compact disc titled “LIGANDS FOR MINERALOCORTICOID RECEPTOR (MR) AND METHODS FOR SCREENING FOR OR DESIGNING MR LIGANDS,” with file title “VAN67 P317 Sequence Listing.ST25.txt,” and is incorporated by reference. This compact disc was created on Nov. 14, 2005 and is twelve kilobytes.

Because of its disease association, MR has been the target of intense pharmaceutical discovery. However, progress toward structural understanding of MR functions has been hampered by the difficulty in obtaining a pure and stable receptor, and as such MR remains the least characterized receptor among the classic steroid hormone receptors. The inventors herein disclose a set of methodology for biochemical and structural analysis of the MR LBD in complex with corticosterone and the SRC1-4 coactivator motif, providing important insights into protein-protein and protein-hormone interactions mediated by MR and its related receptors.

Mechanisms of Coactivator Recognition and Assembly by MR

Co-regulatory proteins such as the SRC family use multiple LXXLL motifs to interact with nuclear receptor LBDs. Taking advantage of the purified MR LBD, the inventors conducted detailed biochemical analysis of MR interactions with coactivators and corepressors using peptide profiling. The results reveal that MR interacts strongly with a specific subset of coactivators, among which are the three SRC coactivators, the two PGC1 coactivators and the DAX1 corepressor. Importantly, these co-regulators have been shown to be expressed in MR target tissues. Both DAX1 and the SRC1a, a spliced isoform of SRC1 that contains the C-terminal SRC1-4 motif, are expressed in discrete regions of brain, including the hypothalamus where MR is highly expressed (Guo et al., 1995; Meijer et al., 2005). PGC1-α and β are also highly expressed in MR target tissues including kidney and heart (Knutti et al., 2000; Puigserver et al., 1998). While the roles of SRC coactivators have been well documented for coactivation of several steroid receptors, the roles of DAX1 and PGC1 in regulating steroid receptors are less characterized. Coexpression of these co-regulators in MR target tissues suggests that MR functions may be regulated through physical interactions with these proteins.

The molecular basis for the selective binding of MR with the above co-regulators is provided by the high resolution structure of the SRC1-4 motif bound to the MR LBD, which reveals specific intermolecular interactions that define the preferential binding of this motif to MR. In the structure, MR uses the conserved charge clamp formed by K785 and E962 to define the general docking mode of the two turn α helix of the SRC1-4 LXXLL motif. The high affinity binding of the SRC1-4 to MR appears to be mediated by the unique charge interactions between the MR K782 residue and the E+7 residue of the SRC1-4 motif since the mutations designed to disrupt the specific hydrogen bond between the E+7 of SRC1-4 and K782 of MR abolish the binding of SRC1-4 to MR in cells (FIG. 4C). Furthermore, mutations of the SRC1-4 motif in the context of full-length SRC1 diminish the ability of SRC1 to coactivate MR, suggesting functional importance of the SRC1-4 binding to MR. Interestingly, the negatively charged residue at the +7 position is also conserved in the 2^{nd} motif of SRC1 and SRC2 (FIG. 3D), and this may help to explain why their binding to MR is comparable to that of the SRC1-4 motif.

Besides the above structural features, MR also contains a second charge clamp formed by residues K791 from helix H3′ and E796 from helix H4 to account for its binding to the third LXXLL motifs of SRC2 (FIG. 2B). The second charge clamp was first observed in the structures of the SRC2-3 motif bound to GR and CAR (Bledsoe et al., 2002; Suino et al., 2004), and was shown to play key roles in specific binding of the 3^{rd} motif from SRC coactivators through hydrogen bonds with the R+2 and D+6 residues within these motifs. Based on the structural conservation and the mutagenesis data shown in FIG. 4D, it is likely that the MR second charge clamp is also involved in the selective binding of the 3^{rd} motif of SRC coactivators.

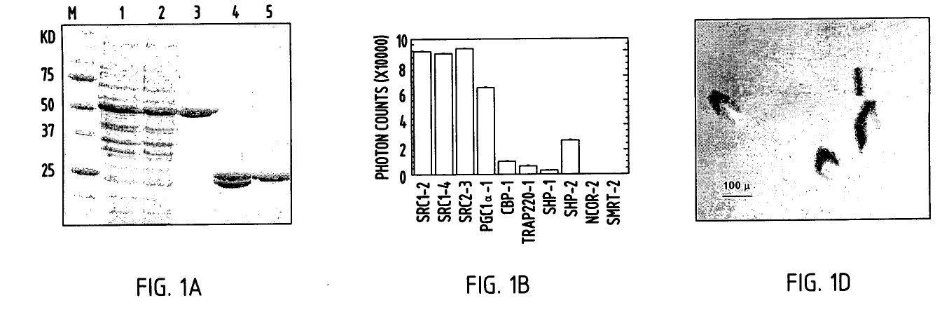

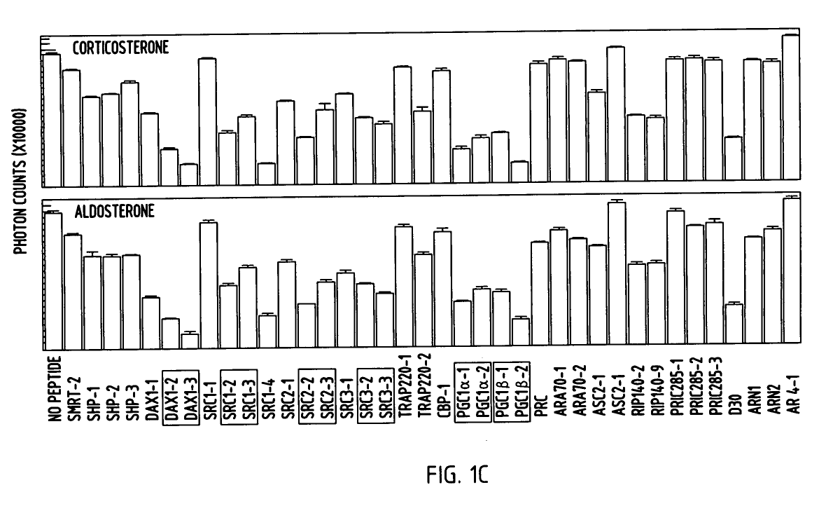

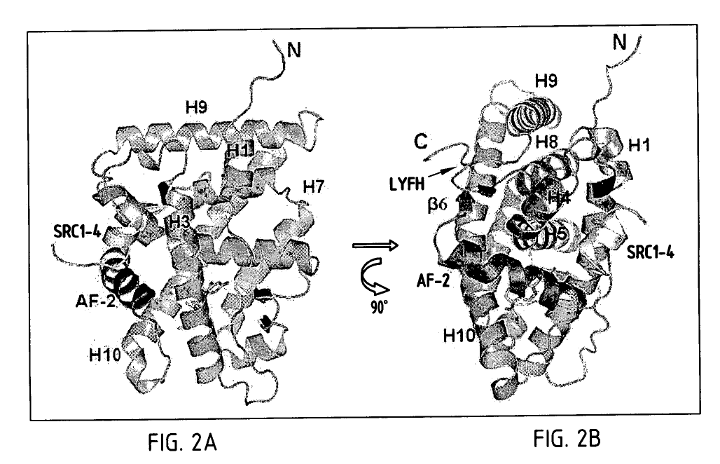

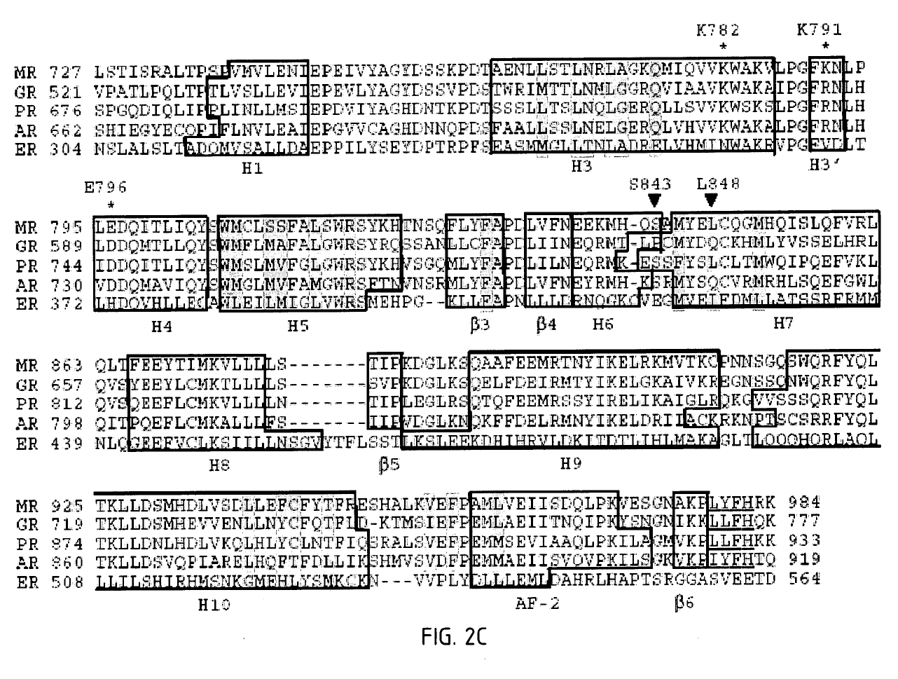

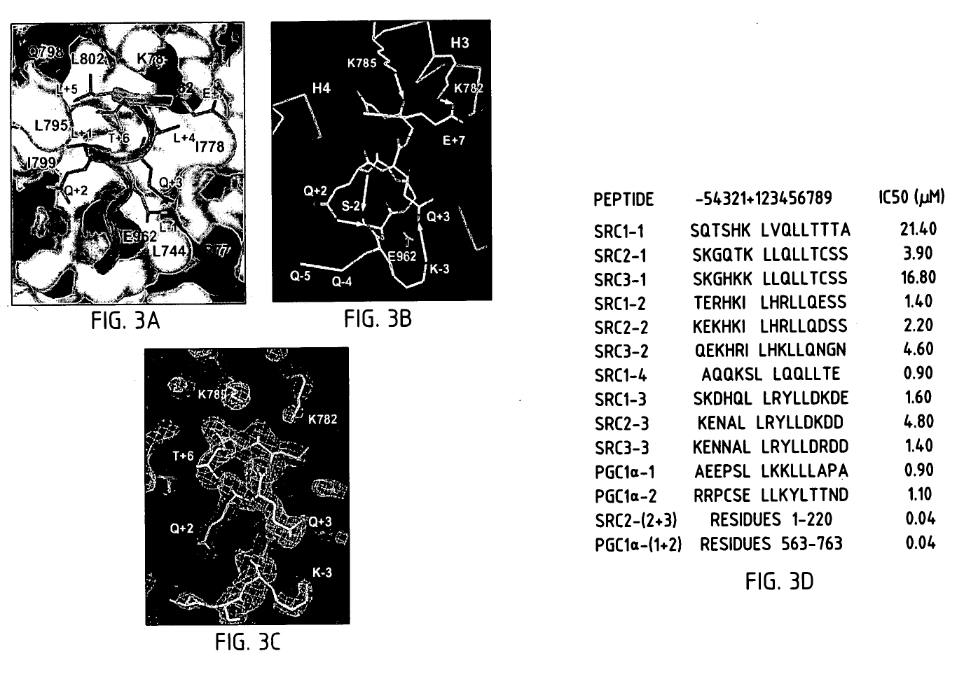

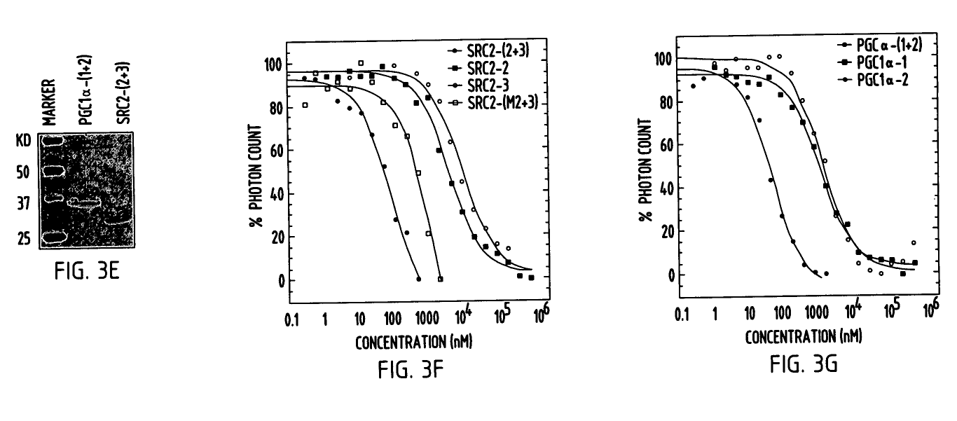

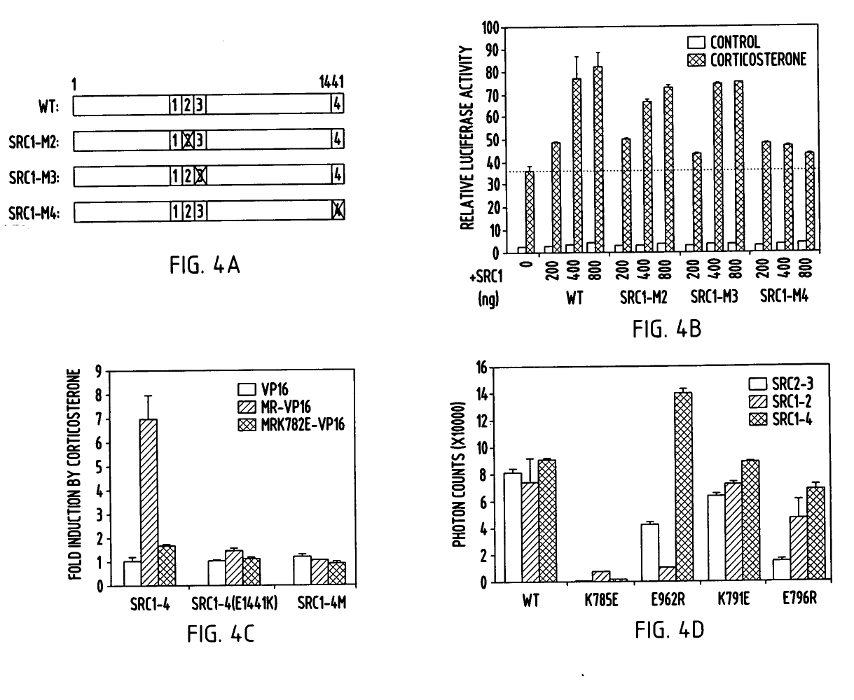

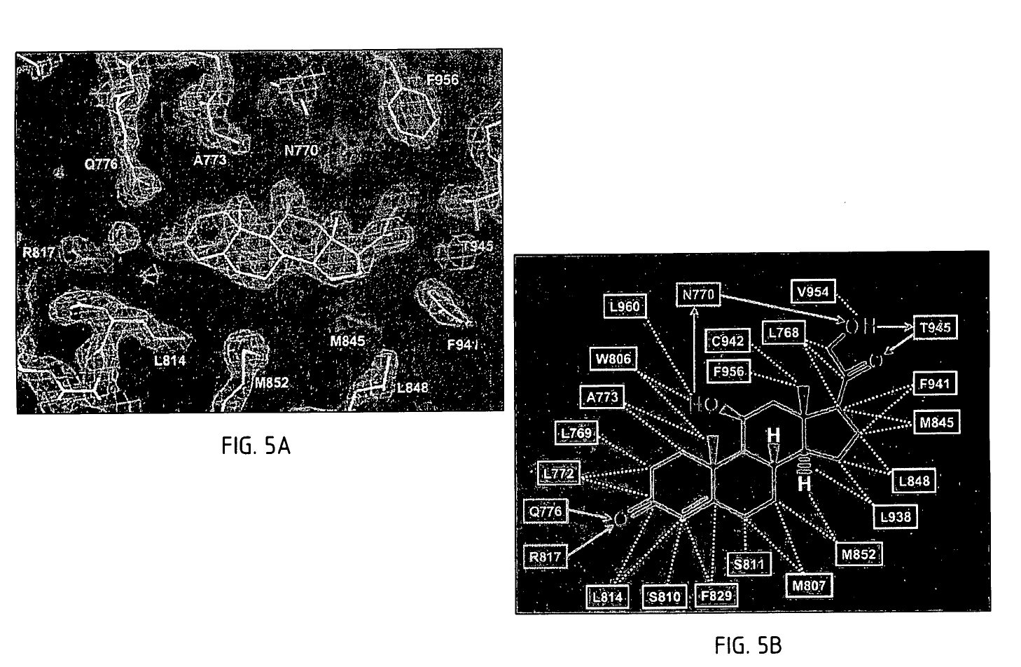

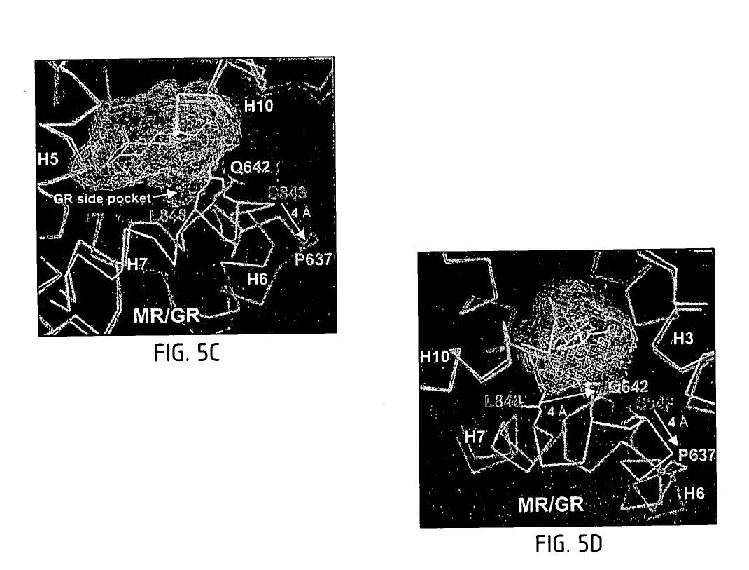

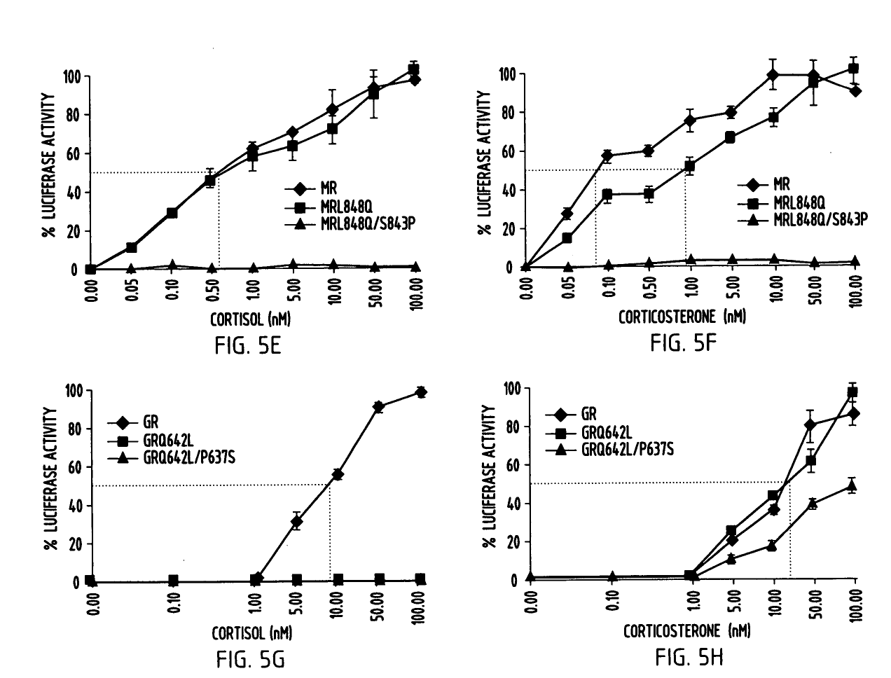

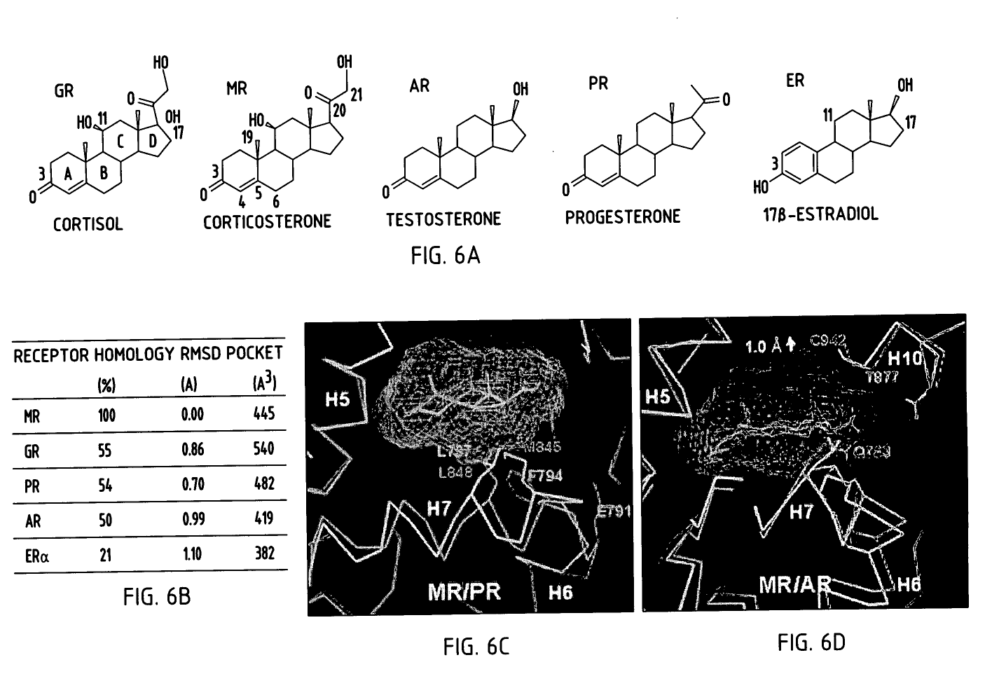

Steroid receptors activate transcription as dimers, and the above data suggest a structural model for the assembly of the MR/coactivator complex, in which SRC coactivators use the 2^{nd} and the 3^{rd} motifs to interact with each LBD of the MR dimer. This mode of MR/coactivator assembly is supported by the inventors' biochemical binding data. Individual motifs from SRC coactivators bind to MR with affinity of 1.0 to 4.0 μM where the 2^{nd} and the 3^{rd} motif in the SRC2 fragment bind to MR with much higher affinity (IC50 of 40 nM), suggesting that both LXXLL motifs bind simultaneously and cooperatively to the MR dimer. Interestingly, DAX1, PGC1α and PGC1β, all contain a pair of LXXLL motifs that interact strongly with MR (FIG. 1C), suggesting that these co-regulators may assemble with MR in a similar dimeric fashion. Since the SRC1-4 motif is essential for the coactivation of MR by SRC1 (FIG. 4B), the dimeric assembly of MR with SRC1 must include the direct docking of the SRC1-4 motif on to the MR coactivator binding site. The facts that both the second charge clamp and K782, the two key features of the coactivator binding site, are conserved between MR and GR, suggest that the mode of coactivator assembly is also conserved in these receptors.

Molecular Basis for the Hormone Specificity of Steroid Receptors

The MR LBD structure is solved last among the classic steroid hormone receptors, and thus provides a final piece of structural puzzle to construct a complete framework for understanding how these steroid receptors distinguish their chemically similar but physiologically distinct hormones. Structural comparisons of MR, GR, PR, AR and ER reveal that these steroid hormone receptors employ three levels of structural mechanisms to define their specific binding to their physiological hormones. The first, and the most critical level of specificity, is the unique hydrogen bond network between the receptor and the bound hormone. All endogenous steroid hormones contain a similar and rigid core chemical structure but have a unique combination of polar groups in the C3, C11 and C17 substitutions (FIG. 6A). Structural inspection of all steroid receptors reveals that the polar groups in the C3, C11 and C17 substitutes of each endogenous hormone are involved in the formation of specific hydrogen bonds with its respective receptor. Because the ligand binding pocket in the steroid receptors is completely enclosed and predominantly hydrophobic, any uncoupled polar groups in the ligand will be a significant penalty to its binding energy. This is particularly illustrated by the inability of cortisol to activate the Q642L G mutant since the C17α hydroxyl of cortisol becomes uncoupled within the mutated GR pocket. Thus, the complete coupling of these steroid polar groups is not only required for the high affinity binding but also provides one critical level of specificity for the receptors to distinguish their hormones. Interestingly, the MR pocket contains a unique polar surface comprised of residues S810 and S811, which are absent from all other steroid receptors. Even though these two residues mediate van der Waal contacts with corticosterone using their Cα and Cβ atoms, the hydroxyl of S810 is within a distance of 3.8-5.1 Å to the C4-6 and C19 atoms of corticosterone. The inventors conceive that synthetic ligands designed to form specific hydrogen bonds with the hydroxyl of S810 will be highly selective for MR.

The second level of specificity that steroid receptors use for hormone recognition is achieved by shape matching between the ligand and its binding pocket. This becomes apparent from structural comparison of MR and GR. Despite that MR is most homologous to GR, the MR LBD structure is in fact most similar to the PR with a compact, steroid shaped ligand binding pocket (FIGS. 6B and 6D), whereas the GR pocket contains a branched side pocket beside the core steroid shape pocket (FIGS. 5C and 5D). The unique topology in the GR pocket appears to rise from the presence of a proline residue (P637) in the linker between helices H6 and H7, which are moved outward for the formation of the GR side pocket. This unique arrangement of the GR side pocket also allows Q642 from helix H7 to make a direct hydrogen bond with C17α hydroxyl groups in glucocorticoids. Remarkably, mutations that swap this residue between MR and GR switch their hormone specificity (FIG. 5E-H). The additional mutation of P637S in GR or S843P in MR severely affects activation of both cortisol and corticosterone, suggesting a critical role of the linker region in the maintenance of intact pocket topology and hormone binding ability. These results are consistent with several previous studies, which demonstrate that the region encompassing helices H6 and H7 is responsible for hormone selectivity of MR, GR, PR and AR (Robin-Jagerschmidt et al., 2000; Rogerson et al., 1999; Vivat et al., 1997). Importantly, the fact that the MR pocket is more compact (or smaller) than the GR pocket helps to explain a longstanding observation: corticosterone and cortisol bind with better affinity to MR than to GR (shown in FIG. 5, 0.1-1.0 nM in MR vs. ˜10 nM in GR; also in (Rogerson et al., 1999)). High affinity binding to GR appears to require large substitutions in the C17α position as observed in fluticasone propionate and mometasone furoate, the active ingredients of marketed anti-allergy medicines Flovent® and Nasonex®.

The third level of hormone specificity appears to be provided by the relative position of the ligand binding pocket within the receptor LBD structure as evident from structural comparisons between MR and AR. The AR pocket appears to be shifted up 1.0 Å toward helices H1 and H3 relative to the MR pocket (FIG. 6C), despite these two receptors having 50% sequence identity in their LBD. The bound androgen also makes a corresponding upward movement to adjust the relocation of the AR pocket. Since the location of the ligand binding pocket is the integrated outcome of all residues that comprise the LBD structure, attempts to change hormone specificity between AR and MR may involve mutations of residues outside of the ligand binding pocket. In fact, it has been shown that the hormone specificity of steroid receptors, such as in the case of ERα and ERβ, may be contributed by residues distal from the pocket, including residues involved in allosteric transmission of ligand binding signal to the receptor dimer for coactivator recruitment and transcriptional activation (Nettles et al., 2004). On the other hand, the MR pocket is aligned exceedingly well with the PR pocket with an RMSD of only 0.70 Å for the Cα atoms of the entire core domain (FIGS. 6B and 6D). Consistent with this structural observation, a single point mutation (S810L) in MR allows the receptor to respond to progesterone, thus providing a molecular basis for the hypertension phenotype exacerbated by pregnancy (Geller et al., 2000).

In summary, the crystal structure of the MR LBD bound to corticosterone and the SRC1-4 LXXLL motif provides important insights into molecular mechanisms that determine the hormone specificity and coactivator assembly by MR. Through peptide binding, SRC1-4 is identified as the most potent coactivator motif that binds to MR and the high resolution structure reveals specific interactions that determine the high affinity binding of SRC1-4 to MR. Importantly, the full-length SRC1 with a defective SRC1-4 motif failed to coactivate MR. In addition, the structure also reveals a compact MR steroid binding pocket and mutations swapping a single pocket residue between MR (L848) and GR (Q642) switch their hormone specificity. Together with the previous structures of other steroid receptors, these results provide a comprehensive framework for understanding the protein-hormone and protein-protein interactions mediated by these receptors. Given the prominent roles of MR in the maintenance of sodium metabolism and blood pressure, these findings also provide a rational template for designing synthetic MR ligands with better selectivity and potency than spironolactone and eplernone. Synthetic MR ligands with higher specificity and affinity may be of great use for the treatment of hypertension and heart failure by reducing the undesired side effects caused by receptor cross reactivity or low potency of the ligands.

Synthetic MR ligands that are agonistic or antagonistic will be valuable tools for understanding MR biology, in addition to their use as pharmaceutical agents for the treatment of MR-related diseases.

The preferred animal for treatment by compounds discovered using the present invention is a mammal, particularly human subjects. By the term “treating,” is meant administering to a subject a pharmaceutical composition comprising an agonist or antagonist of MR whether a steroid hormone or an MR-binding mimic discovered using the screening methods of the invention or designed to de novo using information from the invention.

The pharmaceutical compositions of the present invention comprise an MR ligand combined with pharmaceutically acceptable excipient or carrier, and may be administered by any means that achieve their intended purpose. Amounts and regimens for the administration of such compositions can be determined readily by those of ordinary skill in the clinical art or treatment of the particular diseases. Preferred amounts are described below.

Administration may be by parenteral, subcutaneous, intravenous, intramuscular, intraperitoneal, transdermal, topical, or inhalation routes. Alternatively, or concurrently, administration may be by oral route. The dosage administered will be dependant upon the age, health, and weight of the recipient, kind of concurrent treatment, if any, frequency of treatment, and the nature of the effect desired.

Compositions within the scope of this invention include all compositions wherein the MR receptor ligand is contained in an amount effective to achieve its intended purpose. While individual needs vary, determination of optimal ranges of effective amounts of each component is within the skill of the art. Typical dosages comprise 0.01 to 100 mg/kg/body wt though more preferable dosages may be readily determined without undue experimentation.

As stated above, in addition to the pharmacologically active molecule, the pharmaceutical preparations may contain suitable pharmaceutically acceptable carriers comprising excipients, and auxiliaries which facilitate processing of the active compounds into preparations which can be used pharmaceutically as is well known in the art. Suitable solutions for administration by injection or orally, may contain from about 0.01 to about 99%, active compound(s) together with the excipient.

It will be understood by those who practice the invention and those of ordinary skill in the art that various modifications and improvements may be made to the invention without departing from the spirit of the disclosed concept. The scope of protection afforded is to be determined by the claims and the breadth of interpretation allowed by the law.

### EXAMPLES

The present invention is more particularly described in the following Examples, which are intended as illustrative only, since modifications and variations therein will be apparent to those skilled in the art.

### Example 1

### Experimental Procedures Used in Subsequent Examples

Protein Preparation

The human MR LBD (residues 727-984), containing a C808S mutation, was expressed as a 6×His-GST fusion protein from the expression vector pET24a (Novagen). The fusion protein contains a His6-TAG (MKKGHHHHHHG) at the N terminus and a thrombin protease site between GST and the MR LBD. BL21DE3 cells transformed with this expression plasmid were grown in LB broth at 16° C. to an OD600 of ˜1 and induced with 0.1 mM IPTG and 50 μM corticosterone. Cells were harvested, resuspended in 400 ml extract buffer (50 mM Tris[pH8.0], 150 mM NaCl, 2 M Urea, 10% glycerol) per 24 liters of cells, and passed three times through a French Press with pressure set at 1000 Pa. The lysate was centrifuged at 20,000 rpm for 30 min, and the supernatant was loaded on to a 50 ml glutathione agarose column. The column was washed with 600 ml extract buffer and eluted with 50% buffer B (25 mM Tris [pH8.0], 100 mM NaCl, 20 mM Glutathione, 10% glycerol, 1 M corticosterone). The MR LBD was cleaved overnight with thrombin at a protease/protein ratio of 1:1000 in the cold room. The 6×His-GST tag was removed by a pass through a nickel column. The protein-cofactor complexes were prepared by adding 2-fold excess of SRC1-4 peptide with a sequence of AQQKSLLQQLLTE (SEQ ID NO. 1) to the MR LBD. The ternary complex was further purified by gel filtration (20 mM Tris [pH8.0], 200 mM NaCl, 5 mM DTT, 10% glycerol, 1 μM corticosterone), and filter concentrated to 5 mg/ml. The identities of all purified proteins were confirmed by mass spectrometry. Both human PGC1α (1+2) (residues 1-220) and SRC2-(2+3) (residues 563-763) were expressed as a 6×His-GST fusion protein from the expression vector pET24a (Novagen). The proteins were purified from a Ni-NTA column followed by a Q-Sepharose column.

Crystallization, Data Collection, and Structure Determination

The MR crystals were grown at room temperature in hanging drops containing 3.0 μl of the protein solution and 3.0 μl of well solution containing 0.2 M Sodium Acetate pH 7.9, 24% PEG mme5K, and 25% 1,6-hexanediol. The crystals were directly frozen in liquid nitrogen for data collection. The MR/corticosterone/SRC1-4 crystals formed in the P2₁2₁2₁ space group, with a=44.65 Å, b=72.26 Å, c=81.23 Å, α=β=γ=90° and contains one molecule per crystallographic asymmetric unit. A full 360° data was collected from a single crystal using 1° oscillation by a MAR CCD225 detector at the sector 5ID-B of the Advanced Photon Source, and was processed with HKL2000 (Otwinowski and Minor, 1997). The structures were determined by molecular replacement using the crystal structure of GR LBD (Bledsoe et al., 2002) as a model with the AmoRe program (Navaza et al., 1992). Model building and refinement were carried out with QUANTA (Accelrys Inc) and CNS (Brunger et al., 1998). The pocket volume was calculated with Voidoo using the program default parameters and a probe with radius of 1.20 Å (Kleywegt and Jones, 1994).

Binding Assays

The binding of various peptide motifs to MR was determined by AlphaScreen assays using a hexahistidine detection kit from Perkin-Elmer. MR proteins were prepared as 6×His-GST fusion proteins for the assays. The experiments were conducted with approximately 20 nM receptor LBD and 20 nM of biotinylated SRC2-3 peptide or other coactivator peptides in the presence of 5 μg/ml donor and acceptor beads in a buffer containing 50 nM MOPS, 50 mM NaF, 50 mM CHAPS, and 0.1 mg/ml bovine serum albumin, all adjusted to a pH of 7.4. IC50 values for various coactivator LXXLL motifs were determined from a nonlinear least square fit of the data based on an average of three repeated experiments with standard errors typically less than 10% of the measurements.

The biotinylated peptides that were used in FIG. 1B are listed below in Table 1:

TABLE 1

SEQ
ID

| | | |
|---|---|---|
| Name | Sequence | NO. |

| | | |
|---|---|---|
| SRC2-3 (TIF2) | QEPVSPKKKENALLRYLLDKDDTKD | 2 |
| SRC1-2 | SPSSHSSLTERHKILHRLLQEGSP | 3 |
| SRC1-4 | QKPTSGPQTPQAQQKSLLQQLLTE | 4 |
| PGC1α-1 | AEEPSLLKKLLLAPA | 5 |
| CBP-1 | SGNLVPDAASKHKQLSELLRGGSG | 6 |
| TRAP | GHGEDFSKVSQNPILTSLLQITGN | 7 |
| SHP-1 | PCQGSASHPTILYTLLSPGP | 8 |
| SHP-2 | VAEAPVPSILKKILLEEPNS | 9 |
| NCOR-2 | GHSFADPASNLGLEDIIRKALMGSF | 10 |
| SMRT-2 | QAVQEHASASTNMGLEAIIRKALMGKY | 11 |

The unlabeled peptides that were used in FIG. 1C are listed below in Table 2:

TABLE 2

| | | |
|---|---|---|
| Name | Sequence | SEQ ID NO. |

| | | |
|---|---|---|
| SMRT-2 | ASTNMGLEAIIRKALMGKYDQ | 12 |
| SHP-1 | ASHPTILYTLLSPGP | 13 |
| SHP-2 | APVPSILKKILLEEPNS | 14 |
| SHP-3 | ASQGRLARILLMAST | 15 |
| DAX1-1 | QWQGSILYNMLMSAK | 16 |
| DAX1-2 | PRQGSILYSMLTSAK | 17 |
| DAX1-3 | PRQGSILYSLLTSSK | 18 |
| SRC1-1 | SQTSHKLVQLLTTTA | 19 |
| SRC1-2 | TERHKILHRLLQESS | 20 |
| SRC1-3 | SKDHQLLRYLLDKDE | 21 |
| SRC1-4 | AQQKSLLQQLLTE | 22 |
| SRC2-1 | SKGQTKLLQLLTCSS | 23 |
| SRC2-2 | KEKHKILHRLLQDSS | 24 |
| SRC2-3 | KENALLRYLLDKDD | 25 |
| SRC3-1 | SKGHKKLLQLLTCSS | 26 |
| SRC3-2 | QEKHRILHKLLQNGN | 27 |
| SRC3-3 | KENNALLRYLLDRDD | 28 |
| TRAP220-1 | VSQNPILTSLLQITG | 29 |
| TRAP220-2 | KNIHPMLMNLLDKNP | 30 |
| CBP-1 | ASKHKQLSELLRGGS | 31 |
| PGC1α-1 | AEEPSLLKKLLLAPA | 32 |
| PGC1α-2 | RRPCSELLKYLTTND | 33 |
| PGC1β-1 | VDELSLLQKLLLATS | 34 |
| PGC1β-2 | WAEFSILRELLAQDV | 35 |
| PRC | PREGSSLHKLLTLSR | 36 |
| ARA70-1 | QQQAQQLYSLLGQFN | 37 |
| ARA70-2 | RETSEKFKLLFQSYN | 38 |
| ASC2-1 | TLTSPLLVNLLQSDI | 39 |
| ASC2-2 | REAPTSLSQLLDNSG | 40 |
| RIP140-2 | KQDSTLLASLLQSFS | 41 |
| RIP140-9 | SKSFNVLKQLLLSEN | 42 |
| PRIC285-1 | NADDAILRELLDESQ | 43 |
| PRIC285-2 | NLPPAALRKLLRAEP | 44 |
| PRIC285-3 | FAGDEVLVQLLSGDK | 45 |
| D30 | HSSRLWELLMEAT | 46 |
| ARN1 | YRGAFQNLFQSVR | 47 |
| ARN2 | ASSSWHTLFTAEE | 48 |
| AR4-1 | QPKHFTELYFKS | 49 |

Transient Transfection Assays

Cos-7 cells were maintained in DMEM containing 10% fetal bovine serum (FBS) and were transiently transfected using Lipofectamine 2000 (Invitrogen). 24-Well plates were plated 24 hr prior to transfection (5×104 cells per well). Cells were transfected in Opti-MEM with 400 ng of MMTV-Luc reporter plasmid and 400 ng of receptor expression vector (pRS vector) encoding full-length GR and MR respectively (ATCC). For mammalian two hybrid assays, cells were transfected with 200 ng Gal4-SRC1-4 (residues 1240-1441), 200 ng VP16-MR LBD (residues 727-984), and 200 ng pG5Luc (Promega). For cotransfection of MR and SRC1, 50 ng Gal4-MR LBD was transfected with 200 ng pG5Luc and various amounts of PCR3.1-SRC1 as indicated in the figure legend. 18 hours after transfection, steroids were added in DMEM supplemented with 5% Charcoal/Dextran treated FBS (Hyclone). Cells were harvested 24 hours later for luciferase assays. Luciferase data were normalized to Renilla activity as an internal control.

### Example 2

### The Purified MR LBD Displays Selectivity Toward Coactivator LXXLL Motifs

Similar to GR, the human MR LBD is difficult to express in soluble form due to stability problems, and attempts to purify the wild type MR LBD resulted in mostly aggregated protein. To overcome this problem, the inventors mutated the cysteine residue at position 808 of helix 5 to a serine (C808S), an analogous mutation to the GR F602S mutation, which improved the stability and solubility of the GR LBD (Bledsoe et al., 2002). This point mutated MR LBD appeared to be stable and remained soluble through purification steps in the presence of corticosterone, and was used for biochemical characterization and crystallization throughout this study (FIG. 1A).

To assess the functional activity of the purified MR LBD, the inventors measured the interactions of MR with coactivators and corepressors using a panel of biotinylated peptide motifs (SEQ ID NOS. 2-11) in AlphaScreen assays. As shown in FIG. 1B, the purified MR LBD interacted strongly with various LXXLL motifs from the SRC family of coactivators as well as PGC1 but weakly with CBP, TRAP220, and SHP. In addition, the purified MR LBD failed to interact with LXXXIXXXL corepressor motifs from SMRT or N-COR (NCOR-2 and SMRT-2 in FIG. 1B). This result is consistent with the agonist property of corticosterone, whose binding induces MR to adopt a canonical active conformation that is able to interact with selective coactivators but not with corepressor motifs of SMRT or N-COR.

MR is the least studied member among the classical steroid hormone receptors and its physiological coactivators have not been clearly documented. To gain insights into which coactivator is physiologically relevant to MR, the inventors performed peptide profiling experiments using a panel of 38 unlabeled peptides to compete off the binding of the third LXXLL motif of SRC2 (also known as TIF2/GRIP1) to MR. The sequences of these 38 peptides (SEQ ID NOS. 12-49) shown in Example 1 were selected from endogenous nuclear receptor co-regulators including the SRC family of coactivators, PGC1, SHP, DAX1 and AR coactivator motifs. In the peptide profiling experiment, the amount of each unlabeled peptide used is identical at 500 nM, thus the relative binding affinity of each peptide to MR can be measured by the degree of its inhibition of the binding of the SRC2-3 motif to MR. Consistent with the results above, corepressor motifs did not inhibit SRC2-3 binding to MR but coactivator motifs showed various degrees of inhibition (FIG. 1C). Among these LXXLL motifs, DAX1-3, SRC1-4, PGC1α-1, and PGC1β-2 appear to be the most potent competitors. The strong binding of the SRC1-4 motif and the PGC1 motifs is consistent with previous studies of the binding of LXXLL motifs to oxosteroid receptors (Hultman et al., 2005; Wu et al., 2004).

Peptide profiling is a powerful tool to detect conformational differences of nuclear receptor LBDs with different ligands (Chang et al., 1999). For example, peptide profiling is particularly useful to discern the conformational difference of estrogen receptor (ER) in response to binding of agonist, antagonist and SERMs (selective ER modulators) (Chang et al., 1999). To determine whether there is a conformational difference of MR with a different agonist, the inventors expressed and purified the MR LBD bound with aldosterone for peptide profiling. The result revealed that the aldosterone bound MR has an identical peptide profile as the corticosterone (FIG. 1C), suggesting that MR bound with these two agonists adopts essentially identical conformations. Since the LXXLL motifs of DAX1-3, SRC1-4, PGC1α-1, and PGC1β-2 bind to MR with the highest affinity, these peptides were used for co-crystallization with the corticosterone-bound MR LBD, and the crystals containing the SRC1-4 LXXLL motif were readily obtained (FIG. 1D).

### Example 3

### Structure of the MR LBD/Corticosterone/SRC1-4 Complex

The structure of the MR/corticosterone/SRC1-4 complex was determined to a resolution of 1.95 Å. The statistics of data and the refined structure are listed in Table 1. FIGS. 2A and 2B show the overall structure of the MR/corticosterone/SRC1-4 complex, which is assembled into a monomeric LBD complex in the crystals. Consistent with the sequence homology with GR, PR and AR (FIG. 2C), the MR structure closely resembles the agonist bound structures of these oxosteroid receptors (Bledsoe et al., 2002; Matias et al., 2000; Williams and Sigler, 1998). Specifically, the MR LBD is composed of eleven α helices and four β strands that are folded into a three-layer helical sandwich. The outer layers of helices are formed by helices H1 and H3 on the front and helices H7 and H10 on the back (FIG. 2A). The middle layer of helices (H4, H5, H8 and H9 in FIG. 2B) is clustered at the top half of the domain but is absent from the bottom half, thus creating an interior cavity for the binding of corticosterone. The C-terminal AF-2 helix is positioned in the active conformation by packing tightly against the main domain of the LBD. In this conformation, the AF-2 helix, together with helices H3, H4 and H5, form a charge clamp pocket where the SRC1-4 LXXLL motif is docked. Following the AF-2 helix is an extended strand (β6) that forms a conserved β sheet with a β strand between helices H8 and H9. After this β strand is a highly conserved LYFH motif that forms a hydrophobic core with residues from helices H8, H9 and H10. Both the C-terminal β strand and the LYFH motif appear to be important for ligand binding and receptor activation by stabilizing the canonical LBD fold and tethering the AF-2 helix in the active conformation. Mutations that remove the LYFH motif or residues that form the C-terminal β strand of MR resulted in a receptor that is defective in ligand binding and receptor activation (Couette et al., 1998). Analogous mutations in GR also resulted in an inactive receptor (Zhang et al., 1996), suggesting the packing interactions of the C-terminal β strand and the LYFH motif with the rest of the LBD are important for the activation of these receptors.

### Example 4

### Basis for the Selective Binding of Coactivator LXXLL Motifs

The SRC family of coactivators normally contains three LXXLL motifs and a spliced isoform of SRC1 contains an additional LXXLL motif at its extreme C-terminus (Kalkhoven et al., 1998). This fourth motif of SRC1 (SRC1-4) has been shown to be preferred by GR and PR over other motifs in mammalian two hybrid assays (Needham et al., 2000; Wu et al., 2004). In the peptide profiling experiments (FIG. 1C), the SRC1-4 motif is also the preferred motif by MR, suggesting a conserved mechanism of coactivator recognition by these receptors. The present MR/SRC1-4 structure reveals an unexpected basis for the preferential binding of SRC1-4 to the receptor. In the structure, the LLQQLL sequence of the SRC1-4 motif adopts a two-turn α helix, where the hydrophobic side chains of leucines are directed toward the hydrophobic surface of the coactivator binding site (FIG. 3A). Both ends of the coactivator helix are stabilized by capping interactions with the conserved charge clamp residues E962 from the AF-2 helix and K785 from the end of helix H3, resembling the structure of the GR/TIF2 complex (Bledsoe et al., 2002).

However, the SRC1-4 contains two unique features that define its high affinity binding to MR. The first feature is that the SRC1-4 motif is truncated with a glutamate acid at position +7 (E+7) relative to the first leucines (L+1) in the LXXLL motif (numbering scheme of LXXLL motifs in FIG. 3D). In the structure, the side chain of E+7 forms a direct hydrogen bond with K782 (FIG. 3B). Residue K782 is conserved in MR, GR, AR, and PR (FIG. 2C), and may thus account for the strong binding of the SRC1-4 motif to these receptors (Needham et al., 2000; Wu et al., 2004). The E+7 is also conserved in the 2nd motif of SRC1 and a similar negative charge aspartic acid is presented at the same position of SRC2 (FIG. 3D), which may help to explain why these motifs also interact well with MR (see below). The second feature is the remarkable stability of the SRC1-4 helix in the structure as shown by the excellent electron density for the side chains of two glutamine residues at the center of the LXXLL motif (FIG. 3C). In the structure, Q+3 forms an H-bond with K-3, and Q+2 forms an H-bond with S-2. Residue S-2 also forms a direct hydrogen bond that caps the backbone amide of Q+2 of the LXXLL helix (FIG. 3B). These intramolecular interactions are likely to stabilize the overall helical structure of the SRC1-4 motif. Together, these unique structural features serve as a basis for the high affinity binding of SRC1-4 to MR.

Despite the preferential binding to the SRC1-4 motif, MR also interacted with other SRC LXXLL motifs (FIGS. 1C and 1D). The binding affinities of these motifs to MR were determined by their IC50 values from quantitative competition experiments with unlabeled peptides (FIG. 3D). Consistent with peptide profiling, MR bound to the SRC1-4 motif with the highest affinity (IC50 of 0.9 μM). Interestingly, MR interacted with the 2nd or the 3rd motifs of all three SRC coactivators with approximately the same affinities (IC50 of 1.4 to 4.6 μM in FIG. 3D) but only weakly with the 1st motif of SRC1 and SRC3 (IC50 of 21.4 μM and 16.8 μM, respectively). The binding of the 2nd motif of SRC coactivator can be in part accounted for by the presence of E+7, which forms an H-bond with K782 in the structure. On the other hand, MR, similar to GR, contains a conserved second charge clamp (FIG. 2B), which has been shown to specify the binding of the 3rd motif in the GR/SRC2-3 structure (Bledsoe et al., 2002). To address the role of the MR second charge clamp residues in the binding of various LXXLL motifs, we made mutations in the second charge clamp (K791E and E796R) as well as in the first charge clamp (K785E and E962R) within the MR LBD in the presence of the C808S mutation, and purified these mutated receptors for the binding of three representative LXXLL motifs (SRC1-2, SRC2-3 and SRC1-4). As shown in FIG. 4D, the MR second charge clamp mutation (E796R) significantly decreased the binding of the SRC2-3 motif, which is predicted to form hydrogen bonds with the MR second charge clamp. Correspondingly, the same mutation had little effects on the binding of the SRC1-4 motif, which does not contain complementary residues (R+2 and D+6 in the SRC2-3 motif) to form hydrogen bonds with the MR second charge clamp. The specific effect of the E796R mutation on the binding of SRC2-3 but not SRC1-4 suggests an induced-fit mechanism for the interactions of LXXLL motifs with the second charge clamp, which is formed upon the binding of LXXLL motifs containing residues of R+2 and D+6 (e.g. SRC2-3) but is absent in the binding of LXXLL motifs without the R+2 and D+6 residues such as the SRC1-4 motif. On the other hand, the K785E mutation in the first charge clamp abolished the binding of all three LXXLL motifs as expected. Surprisingly, the E962R mutation in the AF-2 helix only abolished the binding of the SRC1-2 motif, but only partially affected the binding of the SRC2-3 motif and did not affect the binding of the SRC1-4 motif at all (FIG. 4D). The little effect of the E962R mutation in the MR AF-2 in the binding of the SRC1-4 motif is reminiscent of the binding of the PGC1α-1 motif to PPARγ, which is not affected by the E471A mutation in the PPARγ AF-2 helix (Wu et al., 2003). As seen in the SRC1-4 motif, S-2 of the PGC1α-1 motif also interacts with the N-terminal backbone amides of the LXXLL motif in the PPARγ/PGC1α structure and it has been demonstrated that S-2 of PGC1α-1 motif is responsible for the binding of the coactivator motif in the absence of the AF-2 charge clamp residue (the E471A mutation in PPARγ). Sequence alignment of LXXLL motifs (from DAX1, SRC1-4 and PGC1) that bind strongly with MR reveals a conservation of S-2 in these motifs (data not shown), suggesting that S-2 may also play an important role in the binding of these motifs to MR through the capping interaction with the N-terminus of the LXXLL motifs. Together, these results demonstrate that MR contains multiple structural features (1st and 2nd charge clamp and K782) to accommodate the subtle changes of various LXXLL motifs.

The approximately equal binding of the 2^{nd} and the 3^{rd} motif to MR suggests that the SRC family of coactivators may use these two motifs simultaneously to interact with the receptor dimer. Consistent with this idea, a purified SRC2 fragment (SRC2-(2+3)) containing both the 2nd and the 3rd motifs binds to MR with an affinity of 40 nM, which is much higher than the 1-4 μM affinity for the individual motifs (FIGS. 3E and F). The same SRC2 fragment with mutations on the second LXXLL motif decrease the binding affinity more than 10-fold, suggesting that both LXXLL motifs in the SRC2-(2+3) fragment are required for high affinity binding to MR (SRC2-(M2+3) in FIG. 3F). Similar results were also obtained with the PGC1α coactivator (Knutti et al., 2000; Puigserver et al., 1998), where a purified PGC1α fragment (PGC1α-(1+2)) containing both LXXLL motifs binds to MR with much higher affinity than either motif alone (FIG. 3G). Together, these results support a model of MR/coactivator assembly, in which coactivators such as SRC and PGC1 use two LXXLL motifs to interact cooperatively with the dimeric complex of MR.

### Example 5

### The SRC1-4 Motif is Important for Coactivation of MR by SRC1

Among the four LXXLL motifs of the SRC1 coactivator, the SRC1-4 motif binds to MR with the highest affinity (FIGS. 1C and 3D). To validate the functional significance of the SRC1-4 binding to MR, we mutated individual motifs of SRC1-2, SRC1-3 and SRC1-4 within the context of the full-length SRC1 coactivator (FIG. 4A), and tested the ability of these mutated coactivators to potentiate the MR-mediated activation in cell-based assays. FIG. 4B shows that wild type SRC1 significantly elevates the MR-mediated activation at the levels of 400 ng and 800 ng of SRC1 co-transfection plasmids. While mutations on the 2nd or 3rd motif of SRC1 only slightly decreased the SRC1-mediated coactivation, the mutation on the SRC1-4 motif completely abolished the ability of SRC1 to potentiate MR-mediated transcription. These results provide the basis for the functional significance of the SRC1-4 binding to MR and suggest a critical role of the SRC1-4 motif in the coactivation of MR by SRC1.

To probe the molecular mechanisms of the strong interactions of the SRC1-4 motif with MR in vivo, we performed mammalian two hybrid assays using the wild type or mutated SRC1-4 motifs that were fused with the GAL4 DNA-binding domain and the MR LBD that was fused with the VP16 activation domain. In the presence of corticosterone, the SRC1-4 motif induced 7-fold activation of the reporter driven by the GAL4 DNA-binding sites (FIG. 4C), indicating a strong interaction with the MR LBD. The LXXAA mutation in the SRC1-4 motif (SRC1-4M in FIG. 4C) abolished the interaction with MR. In addition to the hydrophobic interactions mediated by the three leucine residues of the LXXLL motif, SRC1-4 also forms a direct hydrogen bond with K782 of MR through the E+7 residue. To assess the importance of this interaction, we made mutations of K782E in MR, or E+7 to K+7 (E1441K) in the SRC1-4 motif, respectively. As expected, the SRC1-4 E1441K mutation failed to interact with MR (FIG. 4C). Conversely, the MR K782E mutation significantly decreased the interaction with SRC1-4 (7 fold to 1.7 fold). Together, these results reveal that the strong interaction of MR with SRC1-4 is due to both hydrophobic binding of its LXXLL motif as shown by the SCR1-4 LXXAA mutation and the specific charged interaction between the flanking E+7 of SRC1-4 and K782 of MR.

### Example 6

### Recognition of Corticosterone by MR

Within the bottom half of the MR LBD is the completely enclosed ligand binding pocket, which scaffold is framed by helices H3, H4, H5, H7, H10, and the first two β strands. The AF-2 helix and its preceding loop also form one side of the pocket. As noted in FIG. 2B, there are 23 residues that line the MR pocket. The total accessible volume of the MR pocket is 445 Å³, comparable to the ligand binding pocket of other steroid hormone receptors (FIG. 6B). The bound corticosterone molecule is completely buried within the MR pocket, whose binding mode can be clearly defined by the exceptional quality of the electron density map (FIG. 5A).

Corticosterone is the physiological mineralocorticoid in rodents and its high affinity binding to MR is readily accounted for by its extensive interactions with the MR pocket residues (FIG. 5B). Within the MR pocket, the bound corticosterone is oriented with its A ring toward the P strands 1 and 2, where the C3 ketone forms a conserved network of hydrogen bond interactions with the side chains of R817 and Q776. The D-ring is oriented toward helix H10 and the AF-2 helix, thus allowing the C20 ketone and C21 hydroxyl groups to form hydrogen bonds with T945 and N770 (FIG. 5B). Residue N770 is conserved in the oxosteroid receptor subfamily and appears to play a key role in ligand recognition and receptor activation. In addition to the H-bond with C21 hydroxyl, N770 also forms close hydrogen bonds with the C-ring 11-hydroxyl and the backbone carbonyl of E955, a residue immediately preceding the AF-2 helix, and thus helps to stabilize this helix in the active conformation.

Besides the above H-bonds with MR, the bound corticosterone also fits nicely into the MR pocket to form an extensive network of hydrophobic interactions. These shape matching interactions include the contact between the C-ring 11-hydroxyl and L960 from the AF-2 helix, and the contacts of the C21 hydroxyl with V954 and F956 from the loop preceding the AF-2 helix (FIG. 5B). The active conformation of the AF-2 is likely stabilized by these complementary protein/ligand interactions. Notably, these interactions are highly conserved among the oxosteroid receptors, thus illustrating a common structural mechanism of hormone-dependent activation for this subfamily of receptors.

### Example 7

### Swapped Mutations Switch the Hormone Specificity of MR and GR

Within the oxosteroid receptor subfamily, MR is most homologous to GR with 60% sequence identity in their LBDs. Consistent with their sequence homology, MR and GR share a similar core LBD structure with an rmsd of 0.86 Å for the Cα atoms from helices 3-12 (FIG. 6B). Despite this similarity, the MR LBD contains three prominent differences from the GR LBD that define the unique characteristics of the MR ligand binding pocket. The first and most prominent difference is the position and orientation of the loop between helices H6 and H7, where there is a serine at residue 843 in MR and a corresponding proline residue (P637) in GR (FIGS. 5C and 5D). The proline residue in the GR loop creates severe geometry constraints in this loop and forces the neighboring GR helices (H6 and the N-terminus of H7) to move 3-4 Å outward from the bound ligand. The outward movement of the GR helices H6 and H7 results in a formation of a GR side pocket that allows a large substitute at the C17α position in GR synthetic agonists such as fluticasone propionate, the active component of marketed drugs Flonase® and Flovent®. The second difference is a leucine residue at MR position 848 but a glutamine residue in the corresponding GR position (Q642). The MR L848 residue forms a close van der Waal contact with the C15 and C16 of corticosterone (FIG. 5B) whereas the Q642 residue of GR runs into the corresponding space occupied by the MR M845 residue and forms a close hydrogen bond with the C17α hydroxyl of dexamethasone or cortisol (Bledsoe et al., 2002). These different interactions help to explain the lack of a hydroxyl group at the C17α position in the MR physiological agonists aldosterone and corticosterone where potent GR agonists contain a hydroxyl or a large substitute in the C17α position. The third difference is the presence of two hydrophilic residues (S810 and S811) in the MR pocket where the corresponding residues are hydrophobic in GR, AR and PR. The unique feature of these two MR residues creates a polar surface in this part of the ligand binding pocket that is compatible to aldosterone, which contains two hydrophilic groups (the C11 oxygen and C18 hydroxyl) that are absent in other steroid hormones. Interestingly, mutations that change S810 to a hydrophobic residue like leucine or methionine allow MR to be activated efficiently by progesterone and cortisone (Geller et al., 2000).

To validate the role of the key MR pocket residues in hormone recognition, the inventors mutated S843 and L848 to the corresponding GR residues (S843P and L848Q) within the context of the full length wild type receptor. The basis for the mutagenesis of these two residues is: L848 is a key pocket residue that distinguishes MR from GR in the recognition of the C17α position of steroids whereas S843 and the corresponding GR residue P637 are located at the center of the short loop between helices H6 and H7 that specify the topology of the MR pocket from the GR pocket. In cell based assays with a MMTV luciferase reporter, the wild type MR was fully activated by corticosterone and cortisol with EC50s of 0.08 nM and 0.6 nM, respectively (FIGS. 5E and 5F). Corticosterone was roughly 10-fold more potent than cortisol. The L848Q mutation appeared to switch the MR ligand preference from corticosterone to cortisol: while activation of MR by cortisol was not affected by the L848Q mutation (FIG. 5E), the potency of corticosterone was decreased by at least 10-fold from an EC50 of <0.1 nM to an EC50 ˜1.0 nM (FIG. 5F). Activation of MR by cortisol and corticosterone was totally abolished by a double mutation of L848Q and S843P, which is located in the linker between helices H6 and H7, and the mutation was predicted to alter the position of the linker and the topology of the pocket. We also performed reverse mutations in the full length GR receptor (P637S and Q642L) and tested them in the same MMTV reporter. Wild type GR was activated by cortisol and corticosterone with a slight preference for cortisol over corticosterone (The EC50 of cortisol is ˜8 nM while the EC50 of corticosterone is ˜20 nM, FIGS. 5G and 5H). The Q642L mutation completely abolished activation by cortisol while activation of corticosterone remained intact, suggesting that the hydrogen bond between Q642 and the C17α hydroxyl is critical for the binding of cortisol to GR. The switched selectivity of the Q642L G mutant for corticosterone over cortisol indicates that this residue is the key that determines the hormone selectivity between MR and GR. These results are consistent with that swapping a small fragment comprising helices H6 and H7 between MR and GR exchanges their hormone specificity (Rogerson et al., 1999). Furthermore, the present MR structure together with the GR structure allows us to determine the key role of L848 of MR (or Q642 of GR) in hormone recognition.

### REFERENCES

Arriza, J. L., Weinberger, C., Cerelli, G., Glaser, T. M., Handelin, B. L., Housman, D. E., and Evans, R. M. (1987). Cloning of human mineralocorticoid receptor complementary DNA: structural and functional kinship with the glucocorticoid receptor. Science 237, 268-275.
Baxter, J. D., Funder, J. W., Apriletti, J. W., and Webb, P. (2004). Towards selectively modulating mineralocorticoid receptor function: lessons from other systems. Mol Cell Endocrinol 217, 151-165.
Beato, M., Herrlich, P., and Schutz, G. (1995). Steroid hormone receptors: many actors in search of a plot. Cell 83, 851-857.
Bledsoe, R. K., Montana, V. G., Stanley, T. B., Delves, C. J., Apolito, C. J., McKee, D. D., Consler, T. G., Parks, D. J., Stewart, E. L., Willson, T. M., et al. (2002). Crystal structure of the glucocorticoid receptor ligand binding domain reveals a novel mode of receptor dimerization and coactivator recognition. Cell 110, 93-105.
Bruner, K. L., Derfoul, A., Robertson, N. M., Guerriero, G., Fernandes-Alnemri, T., Alnemri, E. S., and Litwack, G. (1997). The unliganded mineralocorticoid receptor is associated with heat shock proteins 70 and 90 and the immunophilin FKBP-52. Recept Signal Transduct 7, 85-98.
Brunger, A. T., Adams, P. D., Clore, G. M., DeLano, W. L., Gros, P., Grosse-Kunstleve, R. W., Jiang, J. S., Kuszewski, J., Nilges, M., Pannu, N. S., et al. (1998). Crystallography & NMR system: A new software suite for macromolecular structure determination. Acta Crystallogr D Biol Crystallogr 54, 905-921.
Chang, C., Norris, J. D., Gron, H., Paige, L. A., Hamilton, P. T., Kenan, D. J., Fowlkes, D., and McDonnell, D. P. (1999). Dissection of the LXXLL nuclear receptor-coactivator interaction motif using combinatorial peptide libraries: discovery of peptide antagonists of estrogen receptors alpha and beta. Mol Cell Biol 19, 8226-8239.
Couette, B., Jalaguier, S., Hellal-Levy, C., Lupo, B., Fagart, J., Auzou, G., and Rafestin-Oblin, M. E. (1998). Folding requirements of the ligand-binding domain of the human mineralocorticoid receptor. Mol Endocrinol 12, 855-863.
Funder, J. W. (1997). Glucocorticoid and mineralocorticoid receptors: biology and clinical relevance. Annu Rev Med 48, 231-240.
Funder, J. W. (2003). The role of mineralocorticoid receptor antagonists in the treatment of cardiac failure. Expert Opin Investig Drugs 12, 1963-1969.
Funder, J. W., Pearce, P. T., Smith, R., and Smith, A. I. (1988). Mineralocorticoid action: target tissue specificity is enzyme, not receptor, mediated. Science 242, 583-585.
Galigniana, M. D., Piwien Pilipuk, G., Kanelakis, K. C., Burton, G., and Lantos, C. P. (2004). Molecular mechanism of activation and nuclear translocation of the mineralocorticoid receptor upon binding of pregnanesteroids. Mol Cell Endocrinol 217, 167-179.
Geller, D. S., Farhi, A., Pinkerton, N., Fradley, M., Moritz, M., Spitzer, A., Meinke, G., Tsai, F. T., Sigler, P. B., and Lifton, R. P. (2000). Activating mineralocorticoid receptor mutation in hypertension exacerbated by pregnancy. Science 289, 119-123.
Guo, W., Burris, T. P., and McCabe, E. R. (1995). Expression of DAX-1, the gene responsible for X-linked adrenal hypoplasia congenita and hypogonadotropic hypogonadism, in the hypothalamic-pituitary-adrenal/gonadal axis. Biochem Mol Med 56, 8-13.
Hong, H., Kohli, K., Garabedian, M. J., and Stallcup, M. R. (1997). GRIP1, a transcriptional coactivator for the AF-2 transactivation domain of steroid, thyroid, retinoid, and vitamin D receptors. Molecular & Cellular Biology 17, 2735-2744.
Hultman, M. L., Krasnoperova, N. V., Li, S., Du, S., Xia, C., Dietz, J. D., Lala, D. S., Welsch, D. J., and Hu, X. (2005). The Ligand-dependent Interaction Of Mineralocorticoid Receptor With Coactivator And Corepressor Peptides Suggests Multiple Activation Mechanisms. Mol. Endocrinol.
Kalkhoven, E., Valentine, J. E., Heery, D. M., and Parker, M. G. (1998). Isoforms of steroid receptor co-activator 1 differ in their ability to potentiate transcription by the oestrogen receptor. Embo J 17, 232-243.
Kleywegt, G. J., and Jones, T. A. (1994). Detection, delineation, measurement and display of cavities in macromolecular structures. Acta Cryst D, 178-185.
Knutti, D., Kaul, A., and Kralli, A. (2000). A tissue-specific coactivator of steroid receptors, identified in a functional genetic screen. Mol Cell Biol 20, 2411-2422. Matias, P. M., Donner, P., Coelho, R., Thomaz, M., Peixoto, C., Macedo, S., Otto, N., Joschko, S., Scholz, P., Wegg, A., et al. (2000). Structural evidence for ligand specificity in the binding domain of the human androgen receptor. Implications for pathogenic gene mutations. J Biol Chem 275, 26164-26171.
Meijer, O. C., Kalkhoven, E., van der Laan, S., Steenbergen, P. J., Houtman, S. H., Dijkrnans, T. F., Pearce, D., and de Kloet, E. R. (2005). Steroid receptor coactivator-1 splice variants differentially affect corticosteroid receptor signaling. Endocrinology 146, 1438-1448.
Navaza, J., Gover, S., and Wolf, W. (1992). AMoRe: A new package for molecular replacement. In Molecular Replacement: Proceedings of the CCP4 Study Weekend, E. J. Dodson, ed. (Daresbury, UK, SERC), pp. 87-90.
Needham, M., Raines, S., McPheat, J., Stacey, C., Ellston, J., Hoare, S., and Parker, M. (2000). Differential interaction of steroid hormone receptors with LXXLL motifs in SRC-1a depends on residues flanking the motif. J Steroid Biochem Mol Biol 72, 35-46.
Nettles, K. W., Sun, J., Radek, J. T., Sheng, S., Rodriguez, A. L., Katzenellenbogen, J. A., Katzenellenbogen, B. S., and Greene, G. L. (2004). Allosteric control of ligand selectivity between estrogen receptors alpha and beta: implications for other nuclear receptors. Mol Cell 13, 317-327.
Onate, S. A., Tsai, S. Y., Tsai, M. J., and O'Malley, B. W. (1995). Sequence and characterization of a coactivator for the steroid hormone receptor superfamily. Science 270, 1354-1357.
Otwinowski, Z., and Minor, W. (1997). Processing of x-ray diffraction data collected in oscillation mode. Methods in Enzymology 276, 307-326.
Picard, D., Khursheed, B., Garabedian, M. J., Fortin, M. G., Lindquist, S., and Yamamoto, K. R. (1990). Reduced levels of hsp90 compromise steroid receptor action in vivo. Nature 348, 166-168.
Puigserver, P., Wu, Z., Park, C. W., Graves, R., Wright, M., and Spiegelman, B. M. (1998). A cold-inducible coactivator of nuclear receptors linked to adaptive thermogenesis. Cell 92, 829-839.
Robin-Jagerschmidt, C., Wurtz, J. M., Guillot, B., Gofflo, D., Benhamou, B., Vergezac, A., Ossart, C., Moras, D., and Philibert, D. (2000). Residues in the ligand binding domain that confer progestin or glucocorticoid specificity and modulate the receptor transactivation capacity. Mol Endocrinol 14, 1028-1037.
Rogerson, F. M., Dimopoulos, N., Sluka, P., Chu, S., Curtis, A. J., and Fuller, P. J. (1999). Structural determinants of aldosterone binding selectivity in the mineralocorticoid receptor. J Biol Chem 274, 36305-36311.
Rogerson, F. M., and Fuller, P. J. (2003). Interdomain interactions in the mineralocorticoid receptor. Mol Cell Endocrinol 200, 45-55.
Shiau, A. K., Barstad, D., Loria, P. M., Cheng, L., Kushner, P. J., Agard, D. A., and Greene, G. L. (1998). The structural basis of estrogen receptor/coactivator recognition and the antagonism of this interaction by tamoxifen. Cell 95, 927-937.
Suino, K., Peng, L., Reynolds, R., Li, Y., Cha, J. Y., Repa, J. J., Kliewer, S. A., and Xu, H. E. (2004). The Nuclear Xenobiotic Receptor CAR; Structural Determinants of Constitutive Activation and Heterodimerization. Mol Cell 16, 893-905.
Vivat, V., Gofflo, D., Garcia, T., Wurtz, J. M., Bourguet, W., Philibert, D., and Gronemeyer, H. (1997). Sequences in the ligand-binding domains of the human androgen and progesterone receptors which determine their distinct ligand identities. J Mol Endocrinol 18, 147-160.
Voegel, J. J., Heine, M. J., Tini, M., Vivat, V., Chambon, P., and Gronemeyer, H. (1998). The coactivator TIF2 contains three nuclear receptor-binding motifs and mediates transactivation through CBP binding-dependent and -independent pathways. Embo J 17, 507-519.
Williams, S. P., and Sigler, P. B. (1998). Atomic structure of progesterone complexed with its receptor. Nature 393, 392-396.
Wu, J., Li, Y., Dietz, J., and Lala, D. S. (2004). Repression of p65 transcriptional activation by the glucocorticoid receptor in the absence of receptor-coactivator interactions. Mol Endocrinol 18, 53-62.
Wu, Y., Chin, W. W., Wang, Y., and Burris, T. P. (2003). Ligand and coactivator identity determines the requirement of the charge clamp for coactivation of the peroxisome proliferator-activated receptor gamma. J Biol Chem 278, 8637-8644.
Zhang, S., Liang, X., and Danielsen, M. (1996). Role of the C terminus of the glucocorticoid receptor in hormone binding and agonist/antagonist discrimination. Mol Endocrinol 10, 24-34.

What is claimed is:

1. A method for screening for mineralocorticoid receptor ligands comprising:
isolating an MR-ligand having a high specificity and binding affinity for mineralocorticoid receptor.

2. A method for designing mineralocorticoid receptor ligands comprising: isolating an MR-ligand having a high specificity and binding affinity for mineralocorticoid receptor.

3. A method for designing mineralocorticoid receptor ligands comprising: synthesizing an MR-ligand that forms specific hydrogen bonds with MR residue S810.

4. A pharmaceutical composition comprising: a synthetic mineralocorticoid receptor ligand having high specificity for mineralocorticoid receptor.

5. A pharmaceutical composition comprising: a synthetic mineralocorticoid receptor ligand having high binding affinity for mineralocorticoid receptor.

6. A method for treating a mineralocorticoid receptor-related disease comprising: administering to a subject a pharmaceutical composition having a high specificity for mineralocorticoid receptor.

7. A method for treating a mineralocorticoid receptor-related disease comprising: administering to a subject a pharmaceutical composition having a high binding affinity for mineralocorticoid receptor.

8. The method of claims 6 wherein the pharmaceutical composition is a steroid hormone.

9. The method of claims 6 wherein the pharmaceutical composition is an MR-specific ligand that forms hydrogen bonds with MR residue S810.

10. The method of claims 6 wherein the pharmaceutical composition is an MR-binding mimic.

11. The method of claims 7 wherein the pharmaceutical composition is a steroid hormone.

12. The method of claims 7 wherein the pharmaceutical composition is an MR-specific ligand that forms hydrogen bonds with MR residue S810.

13. The method of claims 7 wherein the pharmaceutical composition is an MR-binding mimic.
